(19) United States
(12) Patent Application Publication
(10) Pub. No.: US 2022/0133923 A1
(43) Pub. Date: May 5, 2022

(54) Modular UVGI Cleaning Keypad

(71) Applicant: Gary Knowls, Montgomery, TX (US)

(72) Inventor: Gary Knowls, Montgomery, TX (US)

(21) Appl. No.: 17/088,902

(22) Filed: Nov. 4, 2020

(51) Int. Cl. A61L 2/10; A61L 2/26

(52) U.S. Cl. CPC A61L 2/10; G06F 3/0202; A61L 2202/11; A61L 2/26

(57) ABSTRACT

The ultraviolet light-cleaning keypad utilizes the sanitizing power of short-wavelength ultraviolet light to clean its usable surface. The keypad can be cleaned between each use to ensure safety for its operators. For ultraviolet light to be cleansing, it must be energetic and very powerful, capable of causing damage to living organisms, including humans. However, the ultraviolet light-cleaning keypad employs protective guards to direct the light and prevent external interference. Furthermore, the light bulb is protected in a secure housing that only lets a slit of light out, minimizing damage if the guards fail.

### BACKGROUND

Ultraviolet germicidal irradiation (UVGI) is a disinfection method that uses short-wavelength ultraviolet (ultraviolet C or UV-C) light to kill or inactivate microorganisms by destroying nucleic acids and disrupting their DNA, leaving them unable to perform vital cellular functions. UVGI is used in a variety of applications, such as food, air, and water purification. Increasingly, it has been employed to sterilize drinking and wastewater since the holding facilities are enclosed and can be circulated to ensure a higher exposure to the UV. In recent years, UVGI has found renewed application in air purifiers. There have been no products available as original equipment or as an aftermarket to address this problem.

In UVGI systems the lamps are shielded or are in environments that limit exposure, such as a closed water tank or closed air circulation system, often with interlocks that automatically shut off the UV lamps if the system is opened for access by humans. For human beings, skin exposure to germicidal wavelengths of UV light can produce rapid sunburn and skin cancer. Exposure of the eyes to this UV radiation can produce extremely painful inflammation of the cornea and temporary or permanent vision impairment, up to and including blindness in some cases. Another potential danger is the UV production of ozone, which can be harmful to one's health. There have been no products available as original equipment or as an aftermarket to address this problem either.

There exists a need for an ultraviolet light-cleaning keypad that is not being met by any known or disclosed device or system of present.

### SUMMARY OF THE INVENTION

The modular ultraviolet light germicidal irradiation cleaning keypad utilizes the sanitizing power of short-wavelength ultraviolet light to clean its usable surface. The keypad can be cleaned between each use to ensure safety for its operators. For ultraviolet light to be cleansing, it must be energetic and very powerful, capable of causing damage to living organisms, including humans. However, the ultraviolet light-cleaning keypad employs protective guards to direct the light and prevent external interference. Furthermore, the light bulb is protected in a secure housing that only lets a slit of light out, minimizing damage if the guards fail.

The removable and configurable modular cartridge comprises an UVC light bulb, housing and runner legs and driver to fit standard keyboards. An embodiment of the disclosure includes the modular cartridge integrated with a standard computer keyboard.

Other aspects and advantages of embodiments of the disclosure will become apparent from the following detailed description, taken in conjunction with the accompanying drawings, illustrated by way of example of the principles of the disclosure herein.

### BRIEF DESCRIPTION OF THE DRAWINGS

FIG. 1 is a view of the keypad with the UVGI bulb housing and guards in accordance with an embodiment of the present disclosure.

FIG. 2 is a view of the keypad and touchscreen implemented with the UVGI bulb housing and guards in accordance with an embodiment of the present disclosure.

FIG. 3 is a flow chart of a method of sanitizing a keyboard in accordance with an embodiment of the present disclosure.

Throughout the description, similar reference numbers may be used to identify similar elements depicted in multiple embodiments. Although specific embodiments of the invention have been described and illustrated, the invention is not to be limited to the specific forms or arrangements of parts so described and illustrated. The scope of the invention is to be defined by the claims appended hereto and their equivalents.

### DETAILED DESCRIPTION

Reference will now be made to exemplary embodiments illustrated in the drawings and specific language will be used herein to describe the same. It will nevertheless be understood that no limitation of the scope of the disclosure is thereby intended. Alterations and further modifications of the inventive features illustrated herein and additional applications of the principles of the inventions as illustrated herein, which would occur to one skilled in the relevant art and having possession of this disclosure, are to be considered within the scope of the invention.

Throughout the present disclosure the term slot is used to refer to an aperture, a slit, an opening, a window and like terms for allowing an emanating of light therefrom. The term ‘slider legs’ refers to runners able to support a housing sliding thereon in a track, in a slot, or a depression. The term ‘slidable relation,’ refers to the motion of the housing on the slider legs.

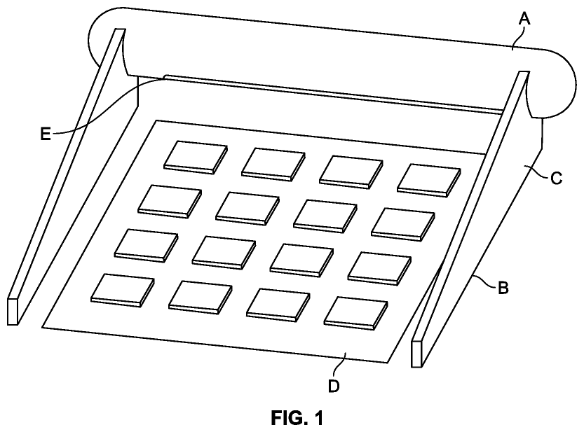

FIG. 1 is a view of the keypad with the UVGI bulb housing and guards in accordance with an embodiment of the present disclosure. Said UV bulb housing A rests on the top of the keypad panel D. On the keypad side of the bulb housing is a slit E for the for light to radiate to the keypad. The side guards C protect against UV exposure and provide three side security. The guards are attached to the keypad by adhesive B. The disclosed device is made as an after market modular and configurable cartridge to adapt to standard computer keyboards. The disclosed device is also integrated with a standard computer keyboard.

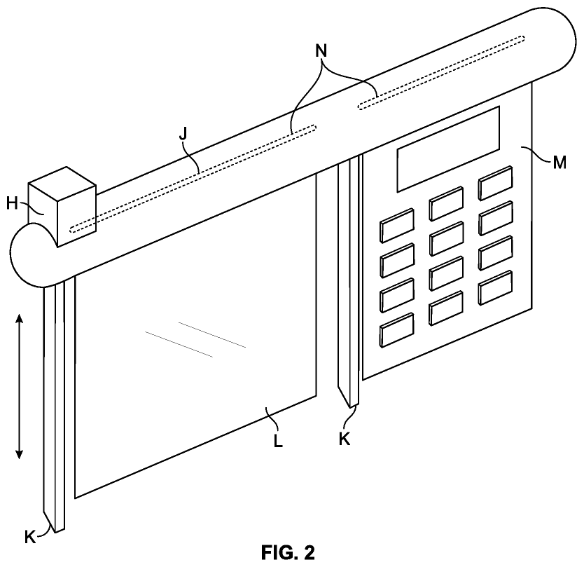

FIG. 2 is a view of the keypad and touchscreen implemented with the UVGI bulb housing and guards in accordance with an embodiment of the present disclosure. The UV bulb housing J holds a drive unit H, and has two slots N, one for the keypad M and one for the touchscreen L. There are safety guards K which attach to each side of the touchscreen. The disclosed device is made as an after market modular and configurable cartridge to adapt to standard computer keyboards. The disclosed device is also integrated with a standard computer keyboard.

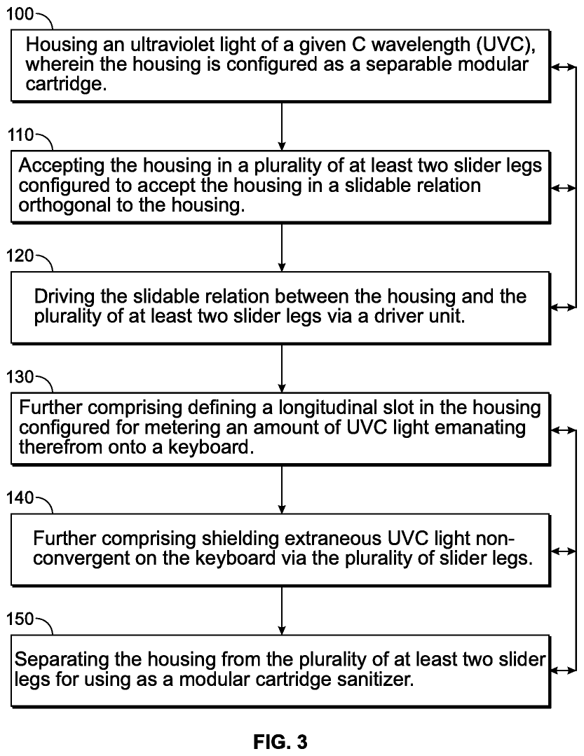

FIG. 3 is a flow chart of a method of sanitizing a keyboard in accordance with an embodiment of the present disclosure. The disclosed method further includes housing 100 an ultraviolet light of a given C wavelength (UVC), wherein the housing is configured as a separable modular cartridge. The method also includes accepting 110 the housing in a plurality of at least two slider legs configured to accept the housing in a slidable relation orthogonal to the housing. The method additionally includes driving 120 the slidable relation between the housing and the plurality of at least two slider legs via a driver unit.

Embodiments of the method are further comprising defining 130 a longitudinal slot in the housing configured for metering an amount of UVC light emanating therefrom onto a keyboard. The method also includes defining a plurality of longitudinal slots in the housing configured for metering an amount of UVC light emanating therefrom onto a keyboard and onto a touch screen.

Embodiments of the method further include adjusting a height of the plurality of at least two slider legs to match a height of a standard keyboard, adjusting a length of the housing to match a standard computer keyboard and adjusting a length of the housing to match a length of a computer keyboard.

Further embodiments of the method are further comprising sliding the housing on the plurality of slider legs at a predetermined rate of motion via a driver unit comprising a stepper motor therein. The method also includes preprogramming a control circuitry in the driver unit for a rate of sliding motion.

Embodiments of the disclosure yet include shielding 140 extraneous UVC light non-convergent on the keyboard via the plurality of slider legs. The embodied methods further include separating 150 the housing from the plurality of at least two slider legs for using as a modular cartridge sanitizer.

Although the operations of the method(s) herein are shown and described in a particular order, the order of the operations of each method may be altered so that certain operations may be performed in an inverse order or so that certain operations may be performed, at least in part, concurrently with other operations. In another embodiment, instructions or sub-operations of distinct operations may be implemented in an intermittent and/or alternating manner.

While the forgoing examples are illustrative of the principles of the present disclosure in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the disclosure be limited, except as by the specification and claims set forth herein.

What is claimed is:

1. A keyboard disinfection device comprising:
a housing for an ultraviolet light of a given C wavelength (UVC), the housing configured as a separable modular cartridge;
a plurality of at least two slider legs configured to accept the housing in a slidable relation orthogonal to the housing; and
a driver unit configured to drive the slidable relation between the housing and the plurality of at least two slider legs.

2. The device of claim 1, further comprising a longitudinal slot defined in the housing, the slot configured to meter an amount of UVC light emanating therefrom onto a keyboard.

3. The device of claim 1, further comprising a plurality of longitudinal slots defined in the housing and configured to meter an amount of UVC light emanating therefrom onto a keyboard and onto a touch screen.

4. The device of claim 1, wherein the plurality of at least two slider legs comprise an adjustable height to match a height of a standard keyboard.

5. The device of claim 1, wherein a length of the housing comprises an adjustable length to match a standard computer keyboard length.

6. The device of claim 1, wherein a length of the plurality of slider legs is adjustable to match a lateral width of a computer keyboard.

7. The device of claim 1, wherein the driver unit comprises a stepper motor configured to slide the housing on the plurality of at least two slider legs at a predetermined rate of motion.

8. The device of claim 1, wherein the driver unit comprises a control circuitry configured to a preprogramming for a rate of sliding motion.

9. The device of claim 1, wherein the plurality of at least two slider legs provide a shield to extraneous UVC light non-convergent on the keyboard.

10. The device of claim 1 wherein the housing is separated from the plurality of at least two slider legs to be used as a modular cartridge sanitizer.

11. A method of sanitizing a keyboard, the method comprising:
housing an ultraviolet light of a given C wavelength (UVC), wherein the housing is configured as a separable modular cartridge;
accepting the housing in a plurality of at least two slider legs configured to accept the housing in a slidable relation orthogonal to the housing; and
driving the slidable relation between the housing and the plurality of at least two slider legs via a driver unit.

12. The method of claim 11, further comprising defining a longitudinal slot in the housing configured for metering an amount of UVC light emanating therefrom onto a keyboard.

13. The method of claim 11, further comprising defining a plurality of longitudinal slots in the housing configured for metering an amount of UVC light emanating therefrom onto a keyboard and onto a touch screen.

14. The method of claim 11, further comprising adjusting a height of the plurality of at least two slider legs to match a height of a standard keyboard.

15. The method of claim 11, further comprising adjusting a length of the housing to match a standard computer keyboard.

16. The method of claim 11, further comprising adjusting a length of the housing to match a length of a computer keyboard.

17. The method of claim 11, further comprising sliding the housing on the plurality of slider legs at a predetermined rate of motion via a driver unit comprising a stepper motor therein.

18. The method of claim 11, further comprising preprogramming a control circuitry in the driver unit for a rate of sliding motion.

19. The method of claim 11, further comprising shielding extraneous UVC light non-convergent on the keyboard via the plurality of slider legs.

20. The method of claim 11, further comprising separating the housing from the plurality of at least two slider legs for using as a modular cartridge sanitizer.
